(19) United States
(12) Plant Patent Application Publication
(10) Pub. No.: US 2026/0033407 P1
(43) Pub. Date: Jan. 29, 2026

(54) CHERRY TREE NAMED 'V690630'

(50) ;

(71) Applicants: Ghassem Tehrani, Guelph (CA); James William Lay, Guelph (CA); Frank Eady, Guelph (CA)

(72) Inventors: Ghassem Tehrani, Guelph (CA); James William Lay, Guelph (CA); Frank Eady, Guelph (CA)

(21) Appl. No.: 18/784,641

(22) Filed: Jul. 25, 2024

(51) Int. Cl. A01H 6/74; A01H 5/00; A01H 5/08

(52) U.S. Cl. CPC A01H 6/7445; A01H 5/00; A01H 5/08
USPC PLT/181

(57) ABSTRACT

A new cherry tree particularly distinguished self-fertility and resistance to rain crack, is disclosed.

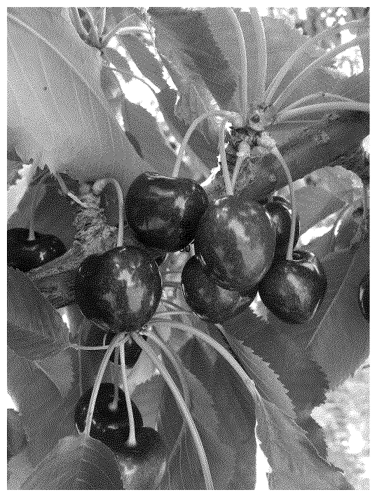

Genus and species: Prunus avium.

Variety denomination: ‘V690630’.

### BACKGROUND OF THE NEW PLANT

The present invention comprises a new and distinct variety of cherry tree, botanically known as Prunus avium and hereinafter referred to by the variety name ‘V690630’. ‘V690630’ originated from a controlled cross conducted in 1969 between the female parent variety ‘Van’ (patent status unknown) and the male parent ‘Stella’ (patent status unknown). ‘V690630’ was bred and developed in Vineland, Ontario, Canada. The resulting seeds were germinated and one resulting seedling named ‘V690630’ was selected. Cherry cultivar ‘V690630’ was found to reproduce true to type in successive generations of asexual propagation via bud wood cuttings.

### SUMMARY

The following are the most outstanding and distinguishing characteristics of this new variety when grown under normal horticultural practices in Wenatchee, Washington.
1. Self-fertile variety; and
2. Resistance to rain crack

### DESCRIPTION OF THE PHOTOGRAPH

This new cherry tree is illustrated by the accompanying photograph show the fruit and leaves of ‘V690630’ from June 2019 on five year old plants grown in Wenatchee, Washington. The colors shown are as true as can be reasonably obtained by conventional photographic procedures.

### DESCRIPTION OF THE NEW VARIETY

The following detailed descriptions set forth the distinctive characteristics of ‘V690630’. The data which define these characteristics was collected in Wenatchee, Washington on five year old trees. All colour determinations were made using 2019 Royal Horticultural Society (R.H.S.) Colour Chart, 6^{th} edition.
Classification:
   Family.—Rosaceae.
   Species.—Prunus avium L.
   Denomination.—‘V690630’.
Parents:
   Female.—Van (patent status unknown).
   Male.—Stella (patent status unknown).
General:
   Height and spread.—3.0 m×2.0 m.
   Cold hardiness.—Zone 5.
   Diseases and pests.—None observed.
   Root description.—Unknown since the plant material was grafted to a rootstock.
   Propagation.—Bud wood in August.
   Growth rate.—1.0 m per season.
Trunk description:
   Size of trunk.—7.5 centimeters at 30 cementer height.
   Bark color.—RHS N200 Dark Grayish Yellow Brown.
   Lenticels.—Yellowish Gray RHS 156.
   Description of dormant shoots (one year-old).—Pubescence: None. Shoot size: Length: 1.0 cm. Width: 0.5 cm. Anthocyanin presence: None. Thickness of shoot at center of middle internode: 1.0 cm. Bark color: RHS N200 Dark Grayish Yellow Brown. Lenticel color: Yellowish Gray RHS 156. Shoot angle: 45 Degrees. Branch internode length: 15 cm.
   Vegetative buds.—Length: 1.0 cm. Width: 0.5 cm. Shape: Oval.
   Color of growing tip of shoot.—RHS 144B Strong Yellow Green.
   Pubescence of shoots tip.—Absent.
   Anthocyanin presence.—Absent.
Foliage description:
   Leaf shape.—Elliptical.
   Leaf division.—Simple.
   Leaf base.—Obtuse with 2 red glands at the base.
   Leaf apex.—Acute.
   Leaf fragrance.—Absent.
   Leaf venation.—Type: Dichotomous. Color: RHS 146A Upper Surface, RHS 146B Lower Surface.
   Leaf margins.—Serrate.
   Leaf arrangement.—Alternate.
   Leaf aspect.—0.5°.
   Leaf attachment.—Petiolate.
   Leaf surface.—Smooth.
   Leaf size.—Length: 13.5 cm. Width: 6.5 cm.
   Leaf color.—Newly expanded leaves: Upper surface: RHS 146B. Lower surface: RHS 146A. Mature leaves: Upper surface: RHS 146A. Lower surface: RHS 146B.
Petioles:
   Length.—3.5 cm.
   Width.—0.2 cm.
   Color.—RHS 146C.
   Anthocyanin.—Absent.
   Texture.—Smooth.
Inflorescence description:
   Blooming period.—April 5 to 12 Osoyoos, British Columbia, Mid season.
   Lastingness of inflorescence.—7 days.
   Flower fragrance.—Cherry like fragrance.
   Flower quantity.—Good.
   Flower type.—Raceme.
   Flower size (diameter).—1.5 cm.
Calyx: Absent.
Sepals:
   Number.—Five.
   Shape.—Conical.
   Margin.—
   Base.—Pointed.
   Apex.—Pointed.
   Color.—Upper surface: RHS 139C. Lower surface: RHS 138C.
   Texture.—Upper surface: Smooth. Lower surfaces: Smooth.
   Length.—0.6 cm.
   Width.—0.5 cm.
Petals:
   Number.—Five.
   Shape.—Obovate.
   Margin.—Regular Margin.
   Base.—Obtuse.
   Apex.—Notches.
   Length.—1.7 cm.
   Width.—0.6 cm.
   Color.—RHS N155B.
Pedicels:
   Length.—3.0 cm.
   Width.—0.2 cm.
   Color.—RHS 143B.
   Texture.—Smooth.
Rachis: Absent.
Reproductive organs:
   Gynoecium.—Pistil: Number: One. Length: 0.6 cm. Style: Color: RHS 143C. Length: 0.6 cm. Width, 0.1 cm. Stigma: Diameter 0.1 cm. Color: RHS 142A. Ovary: Color: RHS 143C.
   Androecium.—Filaments: Color: RHS 155D. Length: 1.0 cm. Anthers: Color: RHS 15C. Pollen: Amount: Large amount. Color: RHS 15C.
Fertility: Self Fertile.
Fruit description:
   Fruit type.—Drupe.
   Fruit shape.—Flat Round.
   Fruit apex.—Round.
   Fruit size.—Length: 2.7 cm. Width: 2.8 cm.
   Fruit firmness.—Firm.
   Fruit set.—Good.
   Fruit stalk.—Length: 4.0 cm. Thickness: 0.2 cm. Surface: Smooth. Color: RHS 143B.
   Fruit bearing.—Yes.
   Number of fruit per cluster.—Three.
   Skin color.—RHS N186A.
   Harvest date.—June 17 Osoyoos, British Colombia 10 days after Bing.
   Skin surface.—Smooth.
   Skin suture.—Medium thick.
   Flesh color.—RHS N186A.
   Juice.—Description: Color: RHS N186A.
   Flesh texture.—Medium.
   Apex end.—Color: RHS N186A.
   Fruit use.—Fresh market.
   Weight.—13 grams.
   Brix.—18.
   Acidity.—0.8 g/100 g.
   Storage life.—6 weeks.
   Productivity.—20,000 lbs. per acre.
Seeds:
   Number per berry.—One.
   Color.—RHS N165B.
   Size.—Length: 1.0 cm. Width: 0.8 cm.
   Shape.—Elliptical.

### COMPARISON WITH PARENTAL LINES AND KNOWN VARIETY

‘V690630’ is distinguished from its parents as described in Table 1.

TABLE 1
Comparison with Parents

| Characteristic | ‘V690630’ | Female parent | Male parent |
|---|---|---|---|
| Self-Fertility | Yes | No | Yes |
| Rain Crack Resistant | Yes | No | No |

‘V690630’ is most similar to the commercial cherry named ‘Bing’ (unpatented). Differences between the two varieties are described in Table 2:

TABLE 2

| Characteristics | ‘V690630’ | ‘Bing’ |
|---|---|---|
| Self-Fertility | Yes | No |

I claim:

1. A new and distinct cultivar of Cherry Tree Named ‘V690630’ as illustrated and described herein.
